(19) United States
(12) Patent Application Publication
(10) Pub. No.: US 2006/0121574 A1
(43) Pub. Date: Jun. 8, 2006

(54) Recombinant method for making multimeric proteins

(75) Inventor: Daniel S. Allison, Seattle, WA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: ICOS CORPORATION, Bothell, WA (US)

(21) Appl. No.: 11/298,020

(22) Filed: Dec. 8, 2005

Related U.S. Application Data
(60) Provisional application No. 60/634,355, filed on Dec. 8, 2004.

(51) Int. Cl. C12P 21/06; C07H 21/04; C12N 5/06; C07K 16/18

(52) U.S. Cl. 435/69.1; 435/70.21; 435/326; 435/320.1; 530/388.1; 536/23.53

(57) ABSTRACT

The present invention relates to methods for making multimeric proteins comprising fusion of two or more cells expressing a single subunit of the multimeric protein to generate a single hybrid cell expressing the fully assembled multimeric protein.

This application claims the benefit of U.S. Provisional Patent Application No. 60/634,355, filed Dec. 8, 2004, incorporated herein by reference.

### FIELD OF THE INVENTION

The present invention relates to a method for making a multimeric protein, wherein the multimeric protein is made by fusing a cell expressing one subunit of the multimeric protein with at least one other cell expressing another subunit of the multimeric protein.

### BACKGROUND OF THE INVENTION

Recombinant proteins are often produced using stably transfected mammalian cell lines. However, random integration of plasmids within chromosomal DNA results in highly variable protein production levels between different transfectants. As a consequence, a large number of transfectants must be screened to identify those with even a moderate level of protein expression. Furthermore, isolation of highly productive cell lines usually requires amplification of the gene encoding the protein of interest. This is typically done either by linking the gene to be expressed directly to a dihydrofolate reductase gene (dhfr) or by co-transfecting a dhfr-negative CHO cell line with both the gene of interest and the dhfr gene, followed by stepwise selection in increasing levels of methotrexate [Kaufman et al., Meth. Enzymol. (1990) 185:537-566]. Constructing highly productive cell lines using this approach is thus a laborious and time-consuming process. In addition, cell lines carrying amplified chromosomal regions are often unstable and lose high level expression after growth in the absence of selective pressure [Weidle et al., Gene (1988) 66:193-203; Fann et al., Biotechnol. Bioeng. (2000) 69:204-212].

Reducing the need for gene amplification would require a many-fold increase in the rate of transcription of each copy of a transfected recombinant gene. Transcription rates of recombinant genes in transfected cell lines are determined both by trans-acting factors specific to the cell line used for expression (e.g., transcription factors), and by cis-acting elements. Cis-acting elements include the promoter and enhancer present in the expression vector, as well as other less-well understood elements present in chromosomal sequences near the site of plasmid integration and which are responsible for position effects [Dillon et al., Trends Genet. (1993) 9:134-137; Hendrich et al., Hum. Mol. Genet. (1995) 4:1765-1777; Hennig, W. Chromosoma (1999) 108:1-9].

In many eukaryotes there are non-coding polynucleotide sequences that enhance gene transcription, for example enhancers, promoters, and locus control regions (LCR), which help regulate tissue specific gene expression [Needham et al., Protein Expression and Purification (1995) 6:124-131]. Recent studies have attempted to make expression vectors which produce increased gene expression by including elements such as a transcriptional enhancer [Khoury et al., Cell (1983), 33:313-314; Blackwood et al., Science (1998), 281:60-63], insulator element [Gerasimova et al., Annu. Rev. Genet. (2001), 35:193-208; West et al., Genes Dev. (2002), 16:271-288], scaffold/matrix attachment region [Bode et al., Crit. Rev. Eukaryot. Gene Expr. (2000), 10:73-90], or transcription termination element [Proudfoot et al., Nature (1986), 322:562-565]. Expression vectors have recently been described that provide increased expression in transfected CHO cells, but depend upon a non-promoter sequence such as an ubiquitous chromatin opening element (UCOE) [Benton et al., Cytotechnology (2002) 38:43-46; International Patent Publication WO 00/05393] or a chicken lysozyme matrix-attachment region (MAR) [Zahn-Zabal et al., J. Biotechnol. (2001) 87:29-42].

Unlike locus control regions (LCR) of a gene, ubiquitous chromatin opening elements are DNA sequences which can enhance gene expression in multiple tissue types (International Patent Publication WO 00/05393). Recent studies have shown that expression of the erythropoietin gene in CHO cells is enhanced when under the control of a UCOE compared to control by a CMV promoter only [WO 00/05393]. One drawback to using UCOEs to facilitate increased gene transcription, however, is their large size, which can be anywhere from 7 kb to 60 kb.

Matrix-attachment regions are DNA sequences that bind nuclear matrices with high affinity and are thought to define boundaries of chromatin domains. MAR elements have been shown to interact with gene enhancers to increase chromatin accessibility and demonstrate enhanced expression of heterologous genes in cultured cell lines [Zahn-Zabal et al., supra]. Thus, MAR elements provide a method for increasing gene expression of heterologous DNA in culture without use of amplification agents.

Recombinant production of multisubunit proteins has proven difficult due to the large amount of DNA and number of plasmids that must be transfected into a single cell to get expression of a functional multisubunit protein. Often the subunits must be expressed in specific ratios to facilitate the optimal assembly of the subunits and subsequent expression of the large protein. Because effective expression of plasmid derived DNA is often limited by insertion site and promoter activity, the protein ratio required by multisubunit proteins is difficult to regulate due to insertion site effects of expression of each gene and control of each subunit gene by various control elements.

Antibodies are multisubunit proteins composed, at their simplest form, of two identical heavy chain and two identical light chain polypeptides joined by disulfide bonds. Antibodies are categorized by class (IgM, IgG, IgD, IgE and IgA) based on the heavy chain gene (CH) they express (μ, γ, δ, ε, α). Antibodies may comprise either one of the two light chain genes (C_{L}), κ or λ. Each heavy chain and light chain polypeptide is encoded by a distinct gene, and the products are translated as separate proteins and assembled in the endoplasmic reticulum of the cell to create the functional multisubunit protein. Recombinant antibody production has been practiced for several years, but initially involved transfection of bacteria or yeast with a single gene encoding either a heavy chain or light chain subunit, and subsequently recovering each protein from its respective cell culture and reassembling the antibody subunits outside the cells. [Cabilly et al., Proc. Natl. Acad. Sci. U.S.A. (1984) 81:3273-7; and U.S. Pat. No. 4,816,567; Wood et al., J. Immunol. (1990)145:3011-16; Simmons et al., J. Immunol. Meth. (2002) 263:133-47].

More recent methods of making recombinant antibodies involve co-transfection of separate vectors respectively expressing a single heavy chain and a light chain into the same cell. This has reportedly been done in mammalian cells [Bender et al., Hum. Antibodies Hybridomas (1993) 4:74-9; Chin et al., Biologicals (2003) 31:45-53; Fan et al., Biol. Chem. (2002) 383:1817-20; Nagahira et al., Immunol. Lett. (1998) 64:139-44] and in insect cells [Hasemann et al., Proc. Natl. Acad. Sci. U.S.A. (1990) 87:3942-6; Guttieri et al., Hybrid Hybridomas (2003) 22:135-45]. While these techniques potentially allow-for assembly of the antibody in the cellular environment, they are subject to difficulties in maintaining expression of two heterologous proteins in an appropriate ratio. For example, in mammalian cells, an antibody heavy chain is often not secreted in the absence of light chain [Struzenberger et al., J. Biotechnol. (1999) 69:215-226].

Monoclonal antibodies are a key therapeutic product in the treatment of numerous conditions and diseases that affect the human population, including autoimmune diseases and cancer. Recombinant monoclonal antibodies are typically made using a co-transfection method as stated above. However, other methods have been described, such as fusion of two monoclonal antibody-producing hybridomas to produce chimeric or bispecific antibodies having a multitude of specificities [Auriol et al., J. Immunol. Meth. (1994) 169:123-33]. Unfortunately, the variability and specificity of the antibodies produced by this technique are too broad when large amounts of single antibody are desired.

Some recent studies have attempted to produce monoclonal antibodies by fusion of two cells, each expressing a different subunit of a multisubunit protein. For example, Norerhaug et al. [Eur. J. Biochem. (2002) 269:3205-10], have attempted to express the secreted isoform of IgA (sIgA), composed of an antibody heavy chain, a light chain, a joining chain (J) and a secretory component (SC). In a multi-step fusion process, a single cell transfected with a plasmid encoding both an antibody heavy chain and a light chain was fused to a cell expressing the antibody J chain, with subsequent fusion of the first fusion product (heavy chain, light chain, J chain in one cell) to a cell expressing the antibody secretory component. While the sIgA molecule is described as having been successfully assembled, this study did not attempt to express the antibody heavy chain gene separately from the light chain gene, which is the primary difficulty in recombinant antibody formation [Struzenberger et al., supra].

Ryll et al [see U.S. Patent Publication No. 20040053363] reports an attempted fusion of two cells separately expressing the heavy and light chain genes with mixed success. This protocol requires addition of amplification agents that can be toxic to the cell, and may not provide an appropriate ratio of the heavy chain to light chain required for significant antibody production in these cells, thereby leading to low multimeric protein recovery. U.S. Pat. Nos. 6,677,138, 6,420,140, 6,207,138, and 5,916,771 describe a method of making a protein using cell fusion techniques, but this protocol also requires addition of amplification agents, and no actual product is demonstrated from this method.

Thus, there remains a need in the art for rapid, large scale production of therapeutically relevant multimeric or multisubunit proteins, such as monoclonal antibodies, wherein the production protocol provides a non-toxic method for growing large numbers of cells and wherein the cells produce each subunit of the multisubunit protein in appropriate ratios.

### DETAILED DESCRIPTION OF THE INVENTION

The present invention provides novel methods for making a recombinant multimeric protein, wherein the production of the multimeric protein in mammalian cells circumvents the need for addition of toxic agents to the cell culture in order to promote gene amplification and increased protein expression.

The present invention contemplates a method for making a multimeric protein comprising the steps of: transfecting a first host cell with a first plasmid comprising a first polynucleotide encoding a first polypeptide of the multimeric protein, wherein the plasmid is not amplified using an amplifiable marker and wherein the plasmid comprises a selectable marker and a regulatory DNA element which provides increased expression of the first polypeptide; transfecting a second host cell with a second plasmid comprising a second polynucleotide encoding a second polypeptide of the multimeric protein, wherein the plasmid is not amplified using an amplifiable marker and wherein the plasmid comprises a selectable marker and a regulatory DNA element which provides increased expression of the second polypeptide; fusing the first host cell with the second host cell to make a cell hybrid, wherein the cell hybrid expresses the first and second polypeptides, and; culturing the cell hybrid in culture media under conditions that permit the expression and association of the polypeptides to form the multimeric protein.

In one embodiment, the method is performed without first selecting for clones expressing the first and second polypeptides prior to the step of fusing the first host cell expressing the first polypeptide and the second host cell expressing the second polypeptide.

In another embodiment, the method optionally comprises one or both of the steps of: selecting a first host cell expressing the first polypeptide by culturing under conditions that permit the expression prior to the fusing step; selecting a second host cell expressing the second polypeptide by culturing under conditions that permit the expression prior to the fusing step.

In another aspect, the method further comprises as many additional transfecting steps as needed to produce a recombinant multisubunit protein having more than two subunits. It is contemplated that the method of the invention optionally comprises one additional transfection step for each additional polypeptide component of the multimeric protein. For example, in one embodiment, multimeric proteins comprising more than two subunits (for example, trimers) are generated using the fusion process described above, wherein polynucleotides encoding the additional protein subunits are inserted into a plasmid, wherein the plasmid is not amplified using an amplifiable marker and wherein the plasmid comprises a selectable marker and a regulatory DNA element which provides increased expression of the encoded polypeptide. This step may be repeated for each subunit of the multimeric protein. The method further comprises the steps of inserting the plasmid into another host cell; fusing the host cell with the host cells fused previously to make an additional cell hybrid, and culturing the cell hybrid in culture media under conditions that permit the expression and association of the polypeptides to form the multimeric protein. The method optionally comprises selecting host cells expressing the additional polypeptide prior to the fusing step. Exemplary trimeric proteins include antibodies of the IgM and IgA subclass, which are composed of a heavy chain, a light chain, and a J chain.

It is contemplated that each plasmid in the transfection step may contain a different selectable marker, including, but not limited to, NeoR (conferring resistance to geneticin), DHFR (allowing cells that lack a functional DHFR gene, such as CHO DG44, to grow in the absence of hypoxanthine and thymidine, and conferring resistance to methotrexate after gene amplification), HisD (conferring resistance to histidinol), PuromycinR (conferring resistance to puromycin), ZeocinR (conferring resistance to zeocin), and GPT (conferring resistance to xanthine-guanine phosphoribosyl-transferase (XGPRT).

By “multimeric” or “multisubunit” is meant a protein comprised of two or more protein subunits. “Multimeric” proteins include heterodimeric or hetero-oligomeric proteins.

By “transformed” or “transfected” is meant that the host cell is modified to contain an exogenous polynucleotide, which can be chromosomally integrated or-maintained in the cell as an episomal element. It is contemplated that in the method of the invention the host cell is transfected in a “transfection step.” The method may comprise multiple transfection steps.

By the terms “a first polypeptide” or “a first polynucleotide” is meant, respectively, the amino acid sequence of, or the nucleotide sequence encoding a single subunit of a multimeric protein that may be expressed by a host cell.

By the terms “a second polypeptide” or “a second polynucleotide” is meant, respectively, the amino acid sequence of, and the nucleotide sequence encoding a single subunit of a multimeric protein that is different from, respectively, the first polypeptide or the first polynucleotide, and which is also expressed by a host cell.

By the term “first host cell” is meant the host cell used to express the subunit encoded by the first polynucleotide, while the term “second host cell” means the host cell used to express the subunit encoded by the second polynucleotide.

It is contemplated that when the multisubunit protein comprises more than a “first polypeptide” and a “second polypeptide”, the additional subunits contemplated will be termed “third polypeptide or third polynucleotide”, and may increase sequentially with each additional subunit. The same terminology criteria may be followed in denominating the host cell and plasmids utilized.

By the term “fusing” or “fusion” of two or more cells is meant a method in which two or more cells are combined to form a single hybrid cell which contains all or part of at least the nucleic acid content of each individual cell. Fusion may be accomplished by any method of combining cells under appropriate conditions well known in the art [See, for example, Harlow & Lane (1988) in Antibodies, Cold Spring Harbor Press, New York]. Known methods for fusing cells include, for example, use of polyethylene glycol (PEG) or Sendai virus. Cells may also be fused using electrofusion [Stoicheva et al., J. Membr. Biol. (1994) 141(2):177-82].

By the term “fusant” or “hybrid cell” is meant a cell formed by combining two or more cells, e.g., by fusion. In the method of the invention, fusants are formed from the fusion of at least two transformed or transfected cells each expressing a different single subunit of a multimeric protein.

By the term “regulatory DNA” is meant DNA sequences, often called cis-acting elements, that are present in chromosomal sequences and that help regulate gene expression. Regulatory DNA includes, but is not limited to, promoters, enhancers, transcriptional enhancers, insulator elements, scaffold/matrix attachment regions, transcription termination elements, ubiquitous chromatin opening elements (UCOE), or other elements present in chromosomal sequences responsible for position effects.

To determine if the cultured cell hybrid permits the expression and association of the multimeric protein, methods for screening both polypeptides and polynucleotides may be performed. The protocol for screening for the protein of interest depends upon the nature of the polypeptide encoded by the inserted polynucleotide and, in some instances, the nature of the host cell. For example, where the recombinant cell contains a polynucleotide that, when expressed, does not produce a secreted product, selection or screening for the presence of cells having the introduced polynucleotide can be accomplished by Northern or Southern blot using a portion of the exogenous polynucleotide sequence as a probe, or by polymerase chain reaction (PCR) using sequences derived from the exogenous polynucleotide sequence as probe. Screening for the expressed, non-secreted polypeptide may be performed using intracellular fluorescent staining by fluorescence activated cell sorting (FACS), through immunoprecipitation methods, or other methods known in the art. If the introduced polynucleotide encodes a secreted polypeptide, the polypeptide may be detected using immunoprecipitation of the polypeptide from the media, or through other detection methods known in the art, such as enzyme-linked immunosorbant assay (ELISA) or FACS.

After fusion of the cells is complete, the recombinant cells containing all the desired polynucleotide sequences can also be identified by detecting expression of a functional multimeric product using, for example, immuno-detection of the product (ELISA, FACS). Alternatively, the expression product can be detected using a bioassay to test for a particular effector function or phenotype conferred by expression of the exogenous sequence(s).

A regulatory DNA contemplated for use in the methods of the invention can be joined to a polynucleotide encoding a monomer of a multimeric protein by any of a variety of other linking nucleotide sequences through well-established recombinant DNA techniques [see Sambrook et al. (2d Ed.; 1989) Molecular Cloning: A Laboratory Manual, Cold Spring Harbor Laboratory, Cold Spring Harbor, N.Y.]. Useful nucleotide sequences for joining to polypeptides include an assortment of vectors, e.g., plasmids, cosmids, lambda phage derivatives, phagemids, and the like, that are well known in the art. The invention also provides a vector including a polynucleotide of the invention and a host cell containing the polynucleotide. In general, the vector contains an origin of replication functional in at least one organism, convenient restriction endonuclease sites, and a selectable marker for the host cell. Useful vectors include, for example, expression vectors, replication vectors, probe generation vectors, sequencing vectors, and retroviral vectors. The host cell can be a eukaryotic cell and can be a unicellular organism or part of a multicellular organism. Large numbers of suitable vectors and promoters are known to those of skill in the art and are commercially available for generating the recombinant constructs of the present invention.

A variety of expression vector/host systems may be utilized to contain and express a polynucleotide contemplated by the invention. These include, but are not limited to, yeast transformed with yeast expression vectors; insect cell systems infected with viral expression vectors (e.g., baculovirus); plant cell systems transfected with virus expression vectors (e.g., Cauliflower Mosaic Virus (CaMV); Tobacco Mosaic Virus (TMV)) or transformed with bacterial expression vectors (e.g., Ti or pBR322 plasmid); or animal cell systems. Mammalian cells that are useful in recombinant protein production include, but are not limited to, VERO cells, HeLa cells, Chinese hamster ovary (CHO) cells, COS cells (such as COS-7), WI38, BHK, HepG2, 3T3, RIN, MDCK, A549, PC12, K562 and HEK 293 cells.

It is contemplated that the methods of the invention utilize a plasmid comprising a transcription regulatory DNA. It is contemplated that the regulatory DNA may be, but is not limited to, a CHEF1 transcription regulatory DNA, a MAR element, or a ubiquitous chromatin opening element (UCOE).

For example, to make a plasmid containing a transcription regulatory DNA, the polynucleotide encoding the Chinese hamster EF1-α regulatory sequence, termed the CHEF1 transcription regulatory DNA (SEQ ID NO: 1 or SEQ ID NO: 2) is inserted into a plasmid for use in either yeast or mammalian expression systems. It is preferable that the CHEF1 regulatory DNA is used in a mammalian expression vector system, and more preferably CHEF1 is used in CHO cells. In another embodiment, the expression vector comprises a UCOE or MAR element to promote increased gene expression. In various embodiments, the method of the invention provides that the first or second plasmid is pNEF5, pDEF14, pDEF2, pDEF10 (described in U.S. Pat. No. 5,888,809), pNEF38, pDEF38 [described in Running Deer et al. (Biotechnol. Prog. (2004) 20:880-889] or pHLEF38 (described herein).

The first host cell and the second host cell of the invention can be cells from the same species. For example, the first host cell and the second host cell can be the same type of cell from the same species. The first host cell and the second host cell can also be different cells from the same species. The first host cell and second host cell may be from different species, for example as described in Dessain et al. [J. Immunol. Meth. (2004) 291:109-22], which describes fusion of a mouse cell line and human B cells to generate antibody producing cells, or Mariani et al. [J. Virol. (2001) 75:3141-51], which describes fusion of human or murine cells with cells from various species. The first host cell and the second host cell can be mammalian cells. The mammalian cells can be CHO cells.

Multimeric proteins in nature may be categorized as homo-oligomeric proteins, large globular proteins, which comprise multiple subunits of the same protein product. These globular proteins include such molecules as collagen, myosin, the resistin family of hormones, and others well known in the art. These types of multimeric proteins are generally expressed from single plasmids and do not require co-transfection of multiple plasmids encoding each subunit. However, many multisubunit proteins are transcribed from different genes and assembled within the cell machinery. Multimeric proteins comprising subunits transcribed from different genes, for example heterodimeric proteins or hetero-oligomeric proteins, contemplated for manufacture through the methods of the invention include, but are not limited to, antibodies, integrins, soluble and membrane-bound forms of MHC (major histocompatibility complex) class I or class II molecules, T cell receptors, the gamma-secretase protease complex, bone morphogenic protein BMP2/BMP7 heterodimeric osteogenic protein, ICE (interleukin-1 converting protein), receptors of the nucleus (e.g., retinoid receptors), heterodimeric cell surface receptors (soluble and membrane forms), tumor necrosis factor (TNF) receptor, and other multimeric proteins in the art.

A multimeric protein made according to the invention can be an antibody product. Antibody products include, but are not limited to, monoclonal antibodies, humanized antibodies, human antibodies, chimeric antibodies, bifunctional/bispecific antibodies, complementary determining region (CDR)-grafted antibodies, Fv fragments, Fab fragments, Fab′ fragments, and F(ab′)₂ fragments.

Antibody products also include CDR sequences or modified CDR sequences, which specifically recognize an antigen of interest. Such antibody products may be chimeric or humanized antibodies, i.e., antibodies that have fully human or largely human antibody structure so as to minimize antigenicity of the antibody itself and otherwise interact with a human immune system in a manner that mimics a true human antibody. Such antibody products may also be human antibodies, which can be produced and identified according to methods described in the art, e.g., in international patent publication WO93/11236, which is incorporated herein by reference in its entirety.

The method of the invention can use DNA encoding an antibody heavy chain or a light chain, or variants or fragments thereof, isolated from a monoclonal or polyclonal antibody which may be produced using techniques common in the art. A monoclonal antibody specific for an antigen of interest may be prepared by using any technique which provides for the production of antibody molecules by continuous cell lines in culture. These include but are not limited to the hybridotna technique originally described by Köhler et al., Nature (1975) 256: 495-497), the more recent human B-cell hybridoma technique [Kosbor et al., Immunol. Today (1983) 4: 72] and the EBV-hybridoma technique [Cole et al., Monoclonal Antibodies and Cancer Therapy, Alan R Liss, Inc., pp. 77-96 (1985), all specifically incorporated herein by reference].

When the hybridoma technique is employed to make a monoclonal antibody, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one can use hybridomas P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one can use hybridomas R210.RCY3, Y3-Ag 1.2.3, IR983F and 4B210; U-266, GM1500-GRG2, LICR-LON-HMy2 and UC729-6. Any of these may be useful in connection with cell fusions as described herein.

The fusant DNAs encoding a monoclonal antibody may be used to produce modified forms of the antibody, such as those that utilize variants or fragment of the antibody sequence, including humanized antibodies, human antibodies, chimeric antibodies, bifunctional/bispecific antibodies, complementary determining region (CDR)-grafted antibodies, Fv fragments, Fab fragments, Fab′ fragments, and F(ab′)₂ fragments. DNAs collectively encoding the modified forms of the antibody may then be used to practice the method of the invention, wherein a fully assembled modified antibody is made by fusion of two or more host cells containing single different subunits of the modified antibody.

For example, techniques developed for the production of “chimeric antibodies”, the splicing of mouse antibody genes to human antibody genes to obtain a molecule with appropriate antigen specificity and biological activity, can be used [Morrison et al., Proc. Natl. Acad. Sci. U.S.A. (1984) 81: 6851-6855; Neuberger et al., Nature (1984) 312: 604-608; Takeda et al., Nature (1985) 314: 452-454] to generate a chimeric antibody.

Antibody fragments that contain the idiotype of the molecule may be generated by known techniques. For example, such fragments include, but are not limited to, the F(ab′)₂ fragment which may be produced by pepsin digestion of the antibody molecule; the Fab′ fragments which may be generated by reducing the disulfide bridges of the F(ab′)₂ fragment, and the two Fab′ fragments which may be generated by treating the antibody molecule with papain and a reducing agent.

Non-human antibodies may be humanized by any methods known in the art. A preferred chimeric or humanized antibody has a human constant region, while the variable region, or at least a CDR, of the antibody is derived from a non-human species. Methods for humanizing non-human antibodies are well known in the art [see U.S. Pat. Nos. 5,585,089 and 5,693,762]. Generally, a humanized antibody has one or more amino acid residues introduced into its framework region from a source which is non-human. Humanization can be performed, for example, using methods described in Jones et al. [Nature (1986) 321:522-525], Riechmann et al., [Nature (1988) 332:323-327] and Verhoeyen et al. [Science (1988) 239:1534-1536], by substituting at least a portion of one or more rodent complementarity-determining regions (CDRs) for the corresponding regions of a human antibody. Numerous techniques for preparing engineered antibodies are described, e.g., in Owens and Young, [J. Immunol. Meth. (1994) 168:149-165]. Further changes can then be introduced into the antibody framework to modulate affinity or immunogenicity.

Polypeptides comprising CDRs are generated using techniques known in the art. Complementarity determining regions are characterized by six polypeptide loops, three loops for each of the heavy or light chain variable regions. The amino acid position in a CDR is defined by Kabat et al., “Sequences of Proteins of Immunological Interest,” U.S. Department of Health and Human Services, (1983), which is incorporated herein by reference. For example, hypervariable regions of human antibodies are roughly defined to be found at residues 28 to 35, from 49-59 and from residues 92-103 of the heavy and light chain variable regions [Janeway and Travers, Immunobiology, 2nd Edition, Garland Publishing, New York, (1996)]. The murine CDRs also are found at approximately these amino acid residues. It is understood in the art that CDRs may be found within several amino acids of the approximate residues set forth above. An immunoglobulin variable region also consists of four “framework” regions surrounding the CDRs (FR1-4). The sequences of the framework regions of different light or heavy chains are highly conserved within a species, and are also conserved between human and murine sequences.

Polypeptides comprising one, two, and/or three CDRs of a heavy chain variable region or a light chain variable region of a monoclonal antibody are generated. For example, based on an antigen-specific monoclonal antibody, polypeptide compositions comprising isolated CDRs are generated. Polypeptides comprising one, two, three, four, five and/or six complementarity determining regions of a monoclonal antibody secreted by a hybridoma are also contemplated. Using the conserved framework sequences surrounding the CDRs, PCR primers complementary to these consensus sequences are generated to amplify the antigen-specific CDR sequence located between the primer regions. Techniques for cloning and expressing nucleotide and polypeptide sequences are well-established in the art [see e.g. Sambrook et al., Molecular Cloning: A Laboratory Manual, 2nd Edition, Cold Spring Harbor, N.Y. (1989)]. The amplified CDR sequences are ligated into an appropriate plasmid. The plasmid comprising one, two, three, four, five and/or six cloned CDRs optionally contains additional polypeptide encoding regions linked to the CDR.

The DNA encoding any of the antibody subunits of the above-described modified antibodies are then transfected into separate host cells which are then fused to generate a fusant expressing a fully assembled modified antibody.

In one simple embodiment, it is contemplated that the first polynucleotide transfected into the first host cell encodes an antibody heavy chain polypeptide or any variant or fragment thereof, while the second polynucleotide transfected into the second host cell encodes an antibody light chain polypeptide or any variant of fragment thereof.

In another embodiment, the invention contemplates a method for making an antibody comprising the steps of: a) transfecting a first host cell with a first plasmid comprising a first polynucleotide encoding a heavy chain polypeptide of the antibody, wherein the plasmid is not amplified using an amplifiable marker and wherein the plasmid comprises a selectable marker and a regulatory DNA element which provides increased expression of the heavy chain polypeptide; b) transfecting a second host cell with a second plasmid comprising a second polynucleotide encoding a light chain polypeptide of the antibody, wherein the plasmid is not amplified using an amplifiable marker and wherein the plasmid comprises a selectable marker and a regulatory DNA element which provides increased expression of the light chain polypeptide; c) fusing the host cells to make a cell hybrid, wherein the cell hybrid expresses the heavy chain polypeptide and the light chain polypeptide; and, d) culturing the cell hybrid in culture media under conditions that permit the expression and association of the heavy chain and the light chain to form the antibody.

In a related embodiment, the method of making an antibody further comprises, before step (c) the step of: (b′) transfecting a third host cell with a third plasmid comprising a third polynucleotide encoding a J chain of the antibody, wherein the plasmid is not amplified using an amplifiable marker and wherein the plasmid comprises a selectable marker and a regulatory DNA element which provides increased expression of the light chain.

It is further contemplated in the method for making an antibody that the fusing step (b) comprises: (i) fusing the transfected host cells obtained from any two of the transfecting steps (a), (b) and (b′) to form an intermediate fusant and (ii) fusing the intermediate fusant with the transfected host cells obtained from the remaining transfecting step (a), (b), or (b′) not fused in (i) to obtain the cell hybrid.

In another embodiment, it is contemplated that the antibody is a Fab fragment, and the heavy chain polypeptide and the light chain polypeptide are fragments capable of permitting expression and association of the Fab fragment.

### EXAMPLES

The following examples are provided to illustrate the invention, but are not intended to limit the scope thereof. Example 1 describes the generation of plasmids comprising the CHEF1 regulatory DNA sequence. Example 2 describes pre-selection fusion of CHO cells each producing a single different antibody heavy chain or light chain subunit. Example 3 describes post-selection fusion of CHO cells each producing a single different antibody heavy chain or light chain subunit.

### Examples 1

### Generation of CHEF1 Driven Plasmids

To obtain high level expression of heterologous genes without using toxic amplification agents, the present invention contemplates use of the Chinese hamster elongation factor-1α (EF-1α) gene 5′ and 3′ flanking sequences. These sequences are described in U.S. Pat. No. 5,888,809, which is hereby incorporated by reference.

Cloning and sequencing the Chinese hamster EF-1α gene. The Chinese Hamster EF-1α gene was cloned from a CHO-K1 genomic library in Lambda FIX II obtained from Stratagene (La Jolla, Calif.) as set out in U.S. Pat. No. 5,888,809 and Running Deer et al. [Biotechnol. Prog. (2004) 20:880-889]. The 18,794 bp of sequence containing the Chinese hamster EF-1α gene and flanking regions has been deposited in Genbank® (Accession number AY188393).

Expression plasmids. The approximately 3.6 kb sequence of the CHEF1 regulatory DNA, including at least the CHEF1 promoter and the 5′ intron, is set out in SEQ ID NO: 1. An approximately 4.1 kb 5 ‘flanking region, recently shown to give increased gene expression [Running Deer et al., (supra)], is set out in SEQ ID NO: 2. The CHEF1 plasmid pDEF14 [Running Deer et al., (supra)] was constructed to include the following segments of DNA: an 11.7 kb DNA fragment from the 5′ flanking region of the CHEF1 gene; 27 bp of synthetic sequence containing HindIII and XbaI sites for insertion of genes to be expressed; a 0.5 kb fragment carrying the phage f1 origin of replication; a 1.8 kb fragment from pSV2-dhfr, which carries a murine dihydrofolate reductase (dhfr) cDNA under the control of promoter/poly(A) addition sequences from the SV40 genome; a 4.2 kb MscI/SalI fragment from the 3′ flanking region of the CHEF1 gene (SEQ ID NO: 3); a 2.2 kb fragment from pBR322 carrying a bacterial origin of replication and the ampicillin resistance gene. To facilitate joining of CHEF1 5′ flanking regions to coding sequences to be expressed, a HindIII site was introduced 15 bp downstream of the acceptor splice site of intron 1 in the CHEF1 gene. The CHEF1 plasmid pNEF5 [Running Deer et al., supra] is identical to pDEF14 except that in pNEF5, the dhfr expression cassette is replaced with a 1.5 kb fragment carrying the neomycin resistance gene (neoR) under the control of SV40 promoter/poly(A) addition sequences. For expression of genes in pDEF14, or pNEF5, a three-way ligation is performed using (1) a HindIII/XbaI fragment carrying the gene of interest, (2) a 737 bp NotI/HindIII fragment from pDEF14, and (3) a ˜19 kb NotI/XbaI vector fragment from the respective vector pDEF14, or pNEF5.

It is contemplated that the size of the 5′ flanking region may be a 4.1 kb fragment from the 5′ flanking region of the CHEF1 gene, including a 6 bp HindIII site at the end of the sequence (SEQ ID NO: 2). For example, CHEF1 vectors pDEF38 and pNEF38 [described in Running Deer et al. (supra)], are identical to pDEF14 and pNEF5, respectively, except that pDEF38 and pNEF38 contain only 4.1 kb of CHEF1 5′ flanking sequence, a more extensive polylinker region, and a 623 base pair PCR-generated XbaI/SalI fragment from the CHEF1 3′ flanking sequence that carries the CHEF1 poly(A) addition sequence, and is positioned on the 3′ side of the polylinker used for insertion of genes to be expressed.

Also contemplated for use in accordance with the invention is CHEF1 vector pHLEF38. Plasmid pHLEF38 is identical to pNEF38 except in pHLEF38, the neo gene is replaced with the histidinol gene (HisD) as the selectable marker. pHLEF38 was constructed via several intermediate plasmids. The first step was to ligate a synthetic linker (5′-AAGCTTCAAGTTATGCTCTAGAATCCGGTAC CTCGAGAAAATGCATGGCAGTCGAC-3′) (SEQ ID NO: 4) which contained HindIII, XbaI, XhoI, NsiI and SalI sites (in that order) into pSL1190 (Pharmacia) cut with HindIII and SalI, creating pSL1190mod. A 4.1 kb NsiI-SalI fragment from pSK/EF1.7 [Running Deer et al., supra] containing the CHEF1 3′ flanking sequence was inserted into the NsiI-SalI sites of pSL1190mod, creating plasmid pSL1190mod/EF13prime.

Next, a 7.29 kb XbaI-XhoI vector fragment from pSL1190mod/EF13prime was ligated to a 638 bp XbaI-BamHI fragment from pDEF38 [Running Deer et al., supra] containing the CHEF1 polyA, and a 3.86 kb BamHI-SalI fragment from pHLEF1 (see below) containing the HisD gene, creating pSL1190mod/HLEF38. This ligation destroys both the internal XhoI and SalI sites, leaving a unique SalI site at the 3′ end of the CHEF1 3′ flank intact in this vector. The 4.2 kb 3′ flanking sequence has also been shown to be important for gene expression [Running Deer et al., supra]

Finally, a 8.65 kb XbaI-SalI fragment from pSL1190mod/HLEF38, containing the CHEF1 polyA, His-D marker cassette and CHEF1 3′ flank, was ligated to a 6.3 kb XbaI-SalI vector fragment from pDEF38 to create pHLEF38.

To create pHLEF1, a 3 kb SfiI-SalI fragment from pREP8 (Invitrogen, San Diego, Calif.), containing the HisD expression cassette, was first ligated with a 5.9 kb XbaI-SalI vector fragment from pNEF1 [U.S. Pat. No. 5,888,809] and an 823 bp SfiI-XbaI fragment from pNEF1. The three-way ligation was necessary because pNEF1 has an additional SfiI site in the CHEF1 promoter, just upstream of the gene insertion site. The plasmid created by this three-piece ligation was named pHLEF1.

### Example 2

### Pre-Selection Fusion: Fusion of CHO Cells Each Comprising Single Antibody Subunit Before Selection of High Producing Cells

To produce a monoclonal antibody IC14, CHO cells expressing the monoclonal antibody IC14 light chain or heavy chain peptides were fused. IC14 is a recombinant chimeric (murine/human) monoclonal antibody (mAb) recognizing human CD14. The murine parent is an Ab designated 28C5 [Leturcq et al., (1996) J. Clin. Invest. 98:1533-38]. IC14 is secreted from Chinese hamster ovary cells as an L₂H_{2γ4} immunoglobulin (Ig).

Generation of CHO Cells Expressing IC14 Light Chain or IC14 Heavy Chain Polypeptide. IC14 heavy chain was inserted into the pDEF14 plasmid while IC14 light chain was inserted into plasmid pNEF5 as follows.

The pDEF14/IC14.HC.IgG4 plasmid consists of pDEF14 in which a 5.82 kb HindIII-XbaI fragment, consisting of the IC14 heavy chain gene with IgG4 3′ flanking sequence (SEQ ID NO: 5), is present within the HindIII-XbaI site of pDEF14, as described in Running Deer et al. (supra). The 5.82 kb sequence contains (1) a HindIII site, (2) an optimized ribosome binding site, (3) the complete coding sequence for the IC14 heavy chain (SEQ ID NO: 6 and 7) and signal sequence, and (4) 4.2 kb of DNA from the 3′ flanking region of the human IgG4 gene [Allison et al, BioProcessing J. March/April 2003:33-40]. Construction of this plasmid was done using standard methods known in the art, and facilitated by the restriction site AgeI, present within the region encoding the CH1 domain of the IgG4 constant sequence, and an NsiI site, present within the region encoding the CH3 domain.

The pNEF5/IC14.LC. plasmid consists of pNEF5 in which a 1.05 HindIII-XbaI fragment, containing the IC14 light chain gene with human kappa 3′ flanking sequence present within the HindIII-XbaI site of pNEF5. The 1.05 kb sequence (SEQ ID NO: 8) contains (1) a HindIII site, (2) an optimized ribosome binding site, (3) the complete coding sequence for the IC14 light chain (SEQ ID NO: 9 and 10) and signal sequence, and (4) 299 bp of DNA from the 3′ flanking region of the human Kappa gene (Allison et al., supra). Plasmid DNA of pNEF5/IC14.LC and pDEF14/IC14.IgG4 was prepared using QIAGEN maxi prep kits (Qiagen, Inc., Valencia, Calif.), according to the manufacturer's instructions.

Transfection of CHO DG44 cells. Two separate transfections were performed, a first transfection introducing only the heavy chain plasmid into CHO cells, and a second transfection introducing only the light chain plasmid into a second set of CHO cells. Before transfection, untransfected CHO DG44 cells were cultured in HT+ medium [DMEM/F12 medium (BioWhittaker, Walkersville, Md.) supplemented with hypoxanthine (0.01 mM), thymidine (0.0016 mM), and 5% -10% dialyzed fetal bovine serum (FBS) obtained either from JRH Biosciences (Lenexa, Kans.) or Hyclone (Logan, Utah)]. Two days prior to transfection, 100% confluent DG44 cells were plated at 1:16 in TI 50 (Corning) flasks in 40 mL HT+ medium. On the day of transfection these cells were approximately 50-60% confluent.

Prior to transfection, 50-100 μg of the plasmid was linearized by digestion with restriction enzymes PvuI or AscI. Sonicated salmon sperm DNA (20 μL) was added prior to ethanol precipitation. The DNA pellet was allowed to air dry briefly and resuspended in 350 μL of sterile, distilled water. Prior to transfection, the DNA was mixed with 450 μL of sterile 2× HeBS (40 mM HEPES, pH 7.0; 274 mM NaCl; 10 mM KCl; 1.4 mM Na₂HPO₄; 12 mM dextrose).

CHO DG44 cells were harvested by trypsinization and quenched with an equal volume of HT+ medium. Cells were counted using a hemocytometer and 2×10⁷ cell per transfection were aliquotted to 15 mL Corning polypropylene tubes. Cells were centrifuged for 5 minutes at 1000 rpm. The medium was aspirated and the cell pellet washed with 10 mL calcium- and magnesium-free phosphate buffered saline (CMF-PBS). Cells were centrifuged again and the PBS aspirated. Each cell pellet was gently resuspended in 0.8 mL of the DNA solution described above. The resuspended cells were transferred to a 0.4 cm gap Gene Pulser cuvette (Bio-Rad, Hercules, Calif.) at room temperature and placed in a Bio-Rad Gene Pulser electroporation apparatus.

Cells were electroporated with a capacitor discharge of 960 μF at 290 Volts. Each cuvette was subjected to one pulse. Time constants varied from 10-11.4 msec. Following electroporation, cells in the cuvettes were allowed to recover at room temperature for 8-10 minutes. Cells were transferred to 15 mL Corning polypropylene tubes containing 10 mL fresh HT+ medium and spun down in a table top centrifuge as above. The medium was aspirated and the cell pellet resuspended in 2 mL HT+ medium, then transferred to a T75 flask containing 20 mL of HT+ medium. Two days following transfection, all cell lines were 90-95% confluent.

Fusion of CHO cells producing IC14 light or IC14 heavy chain Polypeptides. A 37° C. water bath was set up in the cell culture hood. One hundred milliliters (100 mL) of serum free HT+ medium was warmed in two 50 mL Corning polypropylene tubes for 30 minutes prior to cell fusion. One mL of PEG 1500 (Roche PEG 50% w/v in 75 mM HEPES) was warmed in a sterile 1.6 mL microfuge tube.

One T75 flask of each pool of transfected cells, one expressing IC14 heavy chain and one expressing IC14 light chain, was harvested by trypsinization and the cells pooled in a 50 mL Corning polypropylene tube. Cells were spun down in a tabletop centrifuge for 5 minutes at 1000 rpm, washed 3 times with 25 mL warm (37° C.) serum free HT+ medium. The final cell pellet was gently tapped to loosen before addition of PEG.

Using a 1 mL pipette, 1 mL of warmed PEG was added to the cell pellet, incubated in a water bath, over the course of 1 minute. The pellet was then stirred gently with the 1 mL pipette for another minute. With a fresh 1 ml pipet, 1 mL of warmed serum free HT+ medium was added to the mixture, over the course of one minute. Using a 5 mL pipette, 3 mL of warmed serum free HT+ medium was added to the mixture over the course of 3 minutes. Finally, using a 10 mL pipette, 10 mL of warmed serum free HT+ medium was added to the mixture over the course of 3 minutes. The cells were incubated at 37° C. for an additional 5 minutes. The cells were centrifuged as above and the medium aspirated. Cells were resuspended in 2 mL double selection medium HT−/Neo+ [HT+ medium without hypoxanthine/thymidine, plus 800 μg/mL Geneticin® (GIBCO®))]. Cells were plated in two T225 Corning flasks containing 60 mL HT″/Neo+ and allowed to undergo selection for 11 days.

Very few colonies formed in the pre-selection fusion sample (100-150). Those that did form were pooled in a T75 flask, where they reformed colonies instead of forming a lawn. These colonies were pooled in a T25 flask, where again they appeared to reform colonies. This set of colonies was pooled in a fresh T25, and did form a lawn. Cells were slowly expanded to T150 flasks. When the T150 flasks reached 100% confluency, cells were harvested by trypsinization as above and counted on the hemocytometer.

Cells were subcloned in five 96-well flat bottomed plates containing HT−/Neo+ medium, 1 cell per well. Pooled transfected cells were also plated in 3 wells of a 6-well non-TC treated plate, at 1×10⁶ cells perwell in BM18 medium [DME/F12 supplemented with soy hydrolysate and ferrous sulfate [Allison et al, BioProcessing J. March/April 2003:33-40] +10% FBS. The remaining cells were frozen down in HT−/Neo+ medium plus 10% DMSO.

The 6-well plates were incubated with shaking (approximately 75 rpm) at 37° C., 6% CO₂ for 3 days, then shifted to a 34° C., 2% CO_{0.2} shaking incubator for 3 additional days. Supernatants were spun down and filtered with a 0.2 μm syringe filter, then analyzed by Protein A assay (Applied Biosystems, Foster City, Calif.) using the manufacturer's directions to measure assembly of functional antibody.

Results of the Protein A antibody analysis from three replicate transfection experiments showed that the pooled colonies produced titers of approximately 80 μg/mL IC14 antibody, 80 μg/mL IC14 and approximately 60 μg/mL of IC14 antibody, respectively.

Single colonies were then chosen at random from the subcloned plates above, after a two week incubation. These single colonies were expanded to 6 well plates, then transferred to non-tissue culture (TC) treated 6 well plates upon reaching 100% confluency. These plates were grown as described above. Antibody titer from single colonies ranged from approximately 20 μg/mL to approximately 90 μg/mL (see Table 1), with an average titer per colony of approximately 54±19 ug/mL.

These results demonstrate that fusion of the two cells separately transfected with either the heavy chain or light chain of the IC14 antibody produce fully assembled antibody at high titer. This result is contrary to the toxic effect of over-expressed heavy chain on the heavy chain expressing cell observed in other systems.

### Example 3

### Post-Selection Fusion: Fusion of CHO Cells Each Comprising Single Antibody Subunit After Selection of High Producing Cells

It was hypothesized that if cells producing a significant amount of recombinant heavy or light chain protein could be selected before fusion, this would produce larger quantities of assembled product upon fusion of the two high-producing cells. In order to compare the efficacy of antibody chain transfection and recovery of fully assembled recombinant antibody, the ability of CHO cells to produce monoclonal antibody after selection for high expression of the desired protein was tested. This was termed post-selection fusion.

Light chain and heavy chain expressing CHO cells were generated as described in Example 2. Two days following transfection, one T75 flask of each pool of transfected cells, one heavy chain and one light chain, was harvested by trypsinization and replated in two T225 Corning flasks with appropriate selective medium (HT− or HT+/neo+ respectively); HT− medium (same as HT+ medium without the HT supplement); HT+/Neo+ (same as HT+ medium with the addition of 800 μg/mL Geneticin® (GIBCO®)).

A 1:100 dilution of each heavy or light chain transfection was also plated to a 10 cm plate for counting total transfectants. Colonies were allowed to form in selective medium for 10 days. The cell line containing only heavy chain developed close to 15,000 total transfectants, while the light chain cell line yielded 96,000 very small colonies. These colonies were harvested by trypsinization, counted on a hemocytometer and plated in 10 cm plates at 1×10⁶ cells per plate in the appropriate selective medium.

When the 10 cm plates were 100% confluent (a comparable cell number to the 95% confluent T75 used for Pre-Selection Fusion), the fusion procedure was done as described above. Following fusion, cells were plated in a T150 Corning flask in HT−/Neo+ double selection medium for 10 days. Cells were fed fresh medium on day 5.

On day 10, colonies were pooled and replated in fresh T150 Corning flasks. One day later, cells were expanded to two T225 Corning flasks. Two days later, the T225 flasks were 100% confluent. Cells were harvested by trypsinization and counted on the hemocytometer. Cells were subcloned in five 96-well flat bottomed plates, 1 cell per well. Cells were also plated in 6 wells of a 6-well non-TC treated plate, at 1×10⁶ cells per well in BM18+10% FBS medium. The remaining cells were frozen in HT−/Neo+ medium plus 10% DMSO.

The 6-well plates were incubated with shaking (approx. 75 rpm) at 37° C., 6% CO₂ for 3 days, then shifted to a 34° C., 2% CO_{0.2} shaking incubator for 3 additional days. Supernatants were centrifuged and filtered with a 0.2 μm syringe filter, then analyzed by Protein A assay.

Results of the Protein A antibody analysis from three replicate transfection experiments showed that the pooled colonies produced IC14 antibody titers of 32.75 μg/mL (SD=0.65, n=6), which is comparable to titers of the exact same plasmids when co-transfected in CHO cells using a standard electroporation protocol (36.65 μg/mL, SD=5.6, n=6).

After two weeks, single colonies that formed in the 96 well plates were expanded to 6-well TC treated plates and incubated at 37° C. until 100% confluent (about 5 days). These cells were then transferred to 6-well Non-TC treated plates and grown as described above. Antibody titer from single colonies from the post-selection fusion ranged from 0.0 μg/mL in several of the fusants to approximately 60 μg/mL IC14 (see Table 1), with an average titer per colony of approximately 16±17 μg/mL. When only the clones producing antibody were taken into account, the average antibody titer was 21.2±17 μg/mL.

These results show that the fusants coupled after selection of high expressing cells still produced detectable amounts of antibody, though not to the extent of the clones that were not selected before fusion of the two cells (Table 1). This result is contrary to previous theories which suggest that cells overexpressing heavy chain likely would not survive due to the toxicity of the heavy chain protein. However, the pre-selection fusion gave fusants with higher titers which suggests that fusing the transfected cells after selection is disadvantageous in that cell growth may be inhibited by expression of high levels of only the heavy chain or light chain.

TABLE 1

| Pre-selection Fusion | Post-selection Fusion |
|---|---|
| IC14 Antibody Titers | IC14 Antibody Titers |
| 17.15 | 0 |
| 18.5 | 0 |
| 20.13 | 0 |
| 23.36 | 0 |
| 29.21 | 0 |
| 31.81 | 0 |
| 40.94 | 0 |
| 43.33 | 3.82 |
| 45.86 | 4.76 |
| 46.38 | 5.18 |
| 47.64 | 5.32 |
| 50.51 | 5.95 |
| 50.75 | 6.69 |
| 53.3 | 7.55 |
| 54.56 | 8.29 |
| 54.8 | 8.87 |
| 56.02 | 13.31 |
| 57.72 | 19.88 |
| 61.53 | 20.01 |
| 63.02 | 20.16 |
| 68.03 | 20.26 |
| 68.95 | 21.24 |
| 71.83 | 22.09 |
| 72.45 | 28.94 |
| 72.47 | 29.47 |
| 74.88 | 31.07 |
| 78.02 | 40.41 |
| 78.79 | 43.27 |
| 84.66 | 58.77 |
| 88.2 | 62.1 |
| Average: 54.16 ± 20 | Average: 16.25 ± 17.3 |

Overall, these experiments demonstrate that fusion of two separate cells each expressing a different subunit of a heteromultimeric protein, in each case under the control of the CHEF-1 transcription regulatory DNA, generates functional multimeric protein. These results showed that chemical amplification of the gene is not required to get high levels of gene expression before or after fusion of the cells of interest. Moreover, absence of the amplifying agent provides a less toxic environment for the cells to grow and may avoid the high incidence of the fusants eventually becoming low level expressers over time [Strutzenberger et al., J. Biotechnol. (1999) 69:215-26].

Numerous modifications and variations in the invention as set forth in the above illustrative examples are expected to occur to those skilled in the art. Consequently only such limitations as appear in the appended claims should be placed on the invention.

What is claimed is:

1. A method for making a multimeric protein comprising the steps of:
a) transfecting a first host cell with a first plasmid comprising a first polynucleotide encoding a first polypeptide of the multimeric protein, wherein the plasmid is not amplified using an amplifiable marker and wherein the plasmid comprises a selectable marker and a regulatory DNA element which provides increased expression of the first polypeptide;
b) transfecting a second host cell with a second plasmid comprising a second polynucleotide encoding a second polypeptide of the multimeric protein, wherein the plasmid is not amplified using an amplifiable marker and wherein the plasmid comprises a selectable marker and a regulatory DNA element which provides increased expression of the second polypeptide;
c) fusing the host cells to make a cell hybrid, wherein the cell hybrid expresses the polypeptides comprising the multimeric protein, and
d) culturing the cell hybrid in culture media under conditions that permit the expression and association of the polypeptides to form the multimeric protein.

2. The method of claim 1 further comprising an additional transfection step of an additional host cell for each additional polypeptide of the multimeric protein.

3. The method of claim 1 wherein the multimeric protein is a monoclonal antibody, a humanized antibody, a human antibody, a chimeric antibody, a bifunctional/bispecific antibody, a complementarity determining region (CDR)-grafted antibody, a Fv fragment, a Fab fragment, a Fab′ fragment, or a F(ab′)₂ fragment.

4. The method of claim 3 wherein the first polynucleotide encodes an antibody heavy chain polypeptide or variant or fragment thereof.

5. The method of claim 3 wherein the second polynucleotide encodes an antibody light chain polypeptide or variant or fragment thereof.

6. The method of claim 1 wherein the regulatory DNA is CHEF1 transcription regulatory DNA, a MAR element, or a ubiquitous chromatin opening element (UCOE).

7. The method of claim 6 wherein the regulatory DNA is CHEF1 transcription regulatory DNA.

8. The method of claim 1 wherein the first or second plasmid is pNEF5, pDEF14, pDEF2, pDEF10, pDEF38, pNEF38, or pHLEF38.

9. The method of claim 1 wherein the first host cell and the second host cell are the same type of cell.

10. The method of claim 1 wherein the first host cell and the second host cell are mammalian cells.

11. The method of claim 10 wherein the first host cell and second host cell are CHO cells.

12. The method of claim 1 wherein the step of fusing the first host cell and the second host cell is performed without first selecting for host cells expressing the individual polypeptide using appropriate selective media.

13. The method of claim 1 further comprising before step (c), and after step (a) the step of: selecting a first host cell expressing the first polypeptide by culturing under conditions that permit polypeptide expression prior to the fusing step.

14. The method of claim 1 further comprising before step (c), and after step (b) the step of: selecting a second host cell expressing the second polypeptide by culturing under conditions that permit polypeptide expression prior to the fusing step.

15. The method of claim 1 further comprising before step (c), and after step (a) and step (b) the steps of:
selecting a first host cell expressing the first polypeptide by culturing under conditions that permit polypeptide expression prior to the fusing step, and
selecting a second host cell expressing the second polypeptide by culturing under conditions that permit polypeptide expression prior to the fusing step.

16. A method for making an antibody comprising the steps of:
a) transfecting a first host cell with a first plasmid comprising a first polynucleotide encoding a heavy chain polypeptide of the antibody, wherein the plasmid is not amplified using an amplifiable marker and wherein the plasmid comprises a selectable marker and a regulatory DNA element which provides increased expression of the heavy chain polypeptide;
b) transfecting a second host cell with a second plasmid comprising a second polynucleotide encoding a light chain polypeptide of the antibody, wherein the plasmid is not amplified using an amplifiable marker and wherein the plasmid comprises a selectable marker and a regulatory DNA element which provides increased expression of the light chain polypeptide;
c) fusing the host cells to make a cell hybrid, wherein the cell hybrid expresses the heavy chain polypeptide and the light chain polypeptide, and
d) culturing the cell hybrid in culture media under conditions that permit the expression and association of the heavy chain and the light chain to form the antibody.

17. The method of claim 16 further comprising, before step (b) the step of: (b′) transfecting a third host cell with a third plasmid comprising a third polynucleotide encoding a J chain of the antibody, wherein the plasmid is not amplified using an amplifiable marker and wherein the plasmid comprises a selectable marker and a regulatory DNA element which provides increased expression of the light chain.

18. The method of claim 17 wherein the fusing step b) comprises (i) fusing the transfected host cells obtained from any two of the transfecting steps (a), (b) and (b′) to form an intermediate fusant and (ii) fusing the intermediate fusant with the transfected host cells obtained from the remaining transfecting step (a), (b), or (b′) not fused in (i) to obtain the cell hybrid.

19. The method of claim 16, wherein the antibody is an Fab fragment, and the heavy chain polypeptide and the light chain polypeptide are fragments capable of permitting expression and association of the Fab fragment.


SEQUENCE LISTING (10 sequences)

SEQ ID NO: 1 - DNA, 3678 bp, Cricetulus griseus
actagttccaaagatgaattactaaccagtgtttccaaggaaataaatgaaagcagagagattagttctattgctagtgtttcattttcgtatatttcttacaatttctcttgttacaaataggcactagggtatcaagataattttaacgactggctgagaaccctagaaaatctctgtgaaaaagggatttgtgaaatgagagagggtaatgtggccattatagaaaaggcttttgtgtgccttgcatgcatagaccctgtgtttgatctcttaacaccctccttgaccagaaaaagcttctgtggatagaaaatgattagttatatatacttttagggaaacgtagttctggattctttggttacaattaacagaattaagtgcaaacaaagccagaaacctcctgataaatgagaaaacctgcttgtagaaggttgtaaggctctgtaatataggaattaggagaaaagaaacctgtgtggtggggcacgtctgtaatcccagcattgggaagtagaggtagaagattagaaatcaaaggccagcctcagcaacacagtgagtttgaggccaccctgaactacatcaggttctgtctcctttcttttttttttttttttcttttctttttttggtttctctgtgtagttttggagcctatcctggcactagctctgaagagcaggctggcctcgaactcagagatcagccagcctctgctgggattaaaggtatgcaccaccaacgccccaggttttgtctcaaacaaacaaaaataacatcaggaggtggtgagagggctcagtggtcacaggcattctctgcaaagcctgactctgagttggatcctttagagctacatggttgagggaagagaactgactcctggaaggtgtcctctggtccccacacatagctatacacagcatgtgcattcacacacactaaataatgctatttttaaaaaaattaaaaacaacaacagtttgggttgtgaaaactagaactagataataggtaagaatcaagtatcatgtaaatttgctttcaactcatcccaaaatttgttttatatttcagtttttttccttcctagcttgactgtggagtcttgtccggaagcaaatagttcctttgcagatcccacatgtggacaccggacagtaggtcctcaaatgctccttattaggttggttcaataatatcaattgtttgttactaggcagtgatgttgtacatctggaggagatctcttgagcccataatcaggttattaggaataaatactctaaggctaaaaatgtagcttagtgataagagtgcttgcctggtgtgctgagaccctcggttccatctccacaaccccatattccattacaaaataccttttcaccgtccctagcattaagaaacaaaacaacaaagaagtttttctttcttctgagatcctgcccggagaggcatttaaaactggccagggccaaaaaaaaaaaaaaaaaaagaaaaaaaagaaaagaaaacaggctagggccggcatggtggcgcacgcctttaatcccagcacgcaggaggcagaggcagggcggatctctgtgagtttgaggtcagcctggtctacctagtgagtttcagggcacccagggctaaagagactgtctcaaaaacaaaacagccacacaatcagaaccacagcaaaacgcagttatgatccttggaactgtaggaatgacaagcatttaaataataggacgagccatttttgagaagctctgatttcacaagtgtcagggatgggctctgggcgagtaagattgctaatgctggcctctaaatgagaccacgtggagttgattagattcttttcatgttcctcgtgctctatcaaataactgtacccaaatacacacacacacacacacacacacaatgcgcgcacacacaaaatccttttttagcttaagaagcccagaatcagaagtaaagctaactgtgggacttaagtattattctgaacggaactcccagggcgtgaagcgcgcttcaggcttccagagaagcagctggcgctggatggaatgaaccaagaggccagcacaggggcagatccgtcgagctctcggccaccgagctgagcccttaggttctggggctgggaagggtccctaggattgtgcacctctcccgcgggggacaagcaggggatggcggggctgacgtcgggaggtggcctccacgggaagggacacccggatctcgacacagccttggcagtggagtcaggaagggtaggacagattctggacgccctcttggccagtcctcaccgccccacccccgatggagccgagagtaattcatacaaaaggagggatcgccttcgcccctgggaatcccagggaccgtcgctaaattctggccggcctcccagcccggaaccgctgtgcccgcccagcgcggcgggaggagcctgcgcctagggcggatcgcgggtcggcgggagagcacaagcccacagtccccggcggtgggggaggggcgcgctgagcgggggcccgggagccagcgcggggcaaactgggaaagtggtgtcgtgtgctggctccgccctcttcccgagggtgggggagaacggtataaaagtgcggtagtcgcgttggacgttctttttcgcaacgggtttgccgtcagaacgcaggtgagtggcgggtgtggcctccgcgggcccgggctccctcctttgagcggggtcggaccgccgtgcgggtgtcgtcggccgggcttctctgcgagcgttcccgccctggatggcgggctgtgcgggagggcgagggggggaggcctggcggcggccccggagcctcgcctcgtgtcgggcgtgaggcctagcgtggcttccgccccgccgcgtgccaccgcggccgcgctttgctgtctgcccggctgccctcgattgcctgcccgcggcccgggccaacaaagggagggcgtggagctggctggtagggagccccgtagtccgcatgtcgggcagggagagcggcagcagtcgggggggggaccgggcccgcccgtcccgcagcacatgtccgacgccgcctggacgggtagcggcctgtgtcctgataaggcggccgggcggtgggttttagatgccgggttcaggtggccccgggtcccggcccggtctggccagtaccccgtagtggcttagctccgaggagggcgagcccgcccgcccggcaccagttgcgtgcgcggaaagatggccgctcccgggccctgtagcaaggagctcaaaatggaggacgcggcagcccggcggagcggggcgggtgagtcacccacacaaaggaagagggccttgcccctcgccggccgctgcttcctgtgaccccgtggtgtaccggccgcacttcagtcaccccgggcgctctttcggagcaccgctggcctccgctgggggaggggatctgtctaatggcgttggagtttgctcacatttggtgggtggagactgtagccaggccagcctggccatggaagtaattcttggaatttgcccattttgagtttggagcgaagctgattgacaaagctgcttagccgttcaaaggtattcttcgaactttttttttaaggtgttgtgaaaaccaccgctaattcaaatccaacatg

SEQ ID NO: 2 - DNA, 4072 bp, Cricetulus griseus
gtgcatagaaacagatgcaggcaaaacatccacacatataaaacaaaaaattaaaaccaataaaactcctaaacttttggtctttcttgaatcttcaatccctcaggttatgaaataatcatttatgcagtcaaaaatttgccattcttgttgccaggtgtggtgatgattcggggaagcagaagcaggcagatctctgtgaatgaggccagcctggtctacaaagtgagtcccaggacagtcaggcctgttacacagagaaaccttgaaaaaaaaaagataatatgtactgttgtattaccccaatatataaggctaaaccattagaagcacaacactgttaagtacggaaaataatatctagtgtggtacagttactactactataatacactaatatagctgtgggaaactagttccaaagatgaattactaaccagtgtttccaaggaaataaatgaaagcagagagattagttctattgctagtgtttcattttcgtatatttcttacaatttctcttgttacaaataggcactagggtatcaagataattttaacgactggctgagaaccctagaaaatctctgtgaaaaagggatttgtgaaatgagagagggtaatgtggccattatagaaaaggcttttgtgtgccttgcatgcatagaccctgtgtttgatctcttaacaccctccttgaccagaaaaagcttctgtggatagaaaatgattagttatatatacttttagggaaacgtagttctggattctttggttacaattaacagaattaagtgcaaacaaagccagaaacctcctgataaatgagaaaacctgcttgtagaaggttgtaaggctctgtaatataggaattaggagaaaagaaacctgtgtggtggggcacgtctgtaatcccagcattgggaagtagaggtagaagattagaaatcaaaggccagcctcagcaacacagtgagtttgaggccaccctgaactacatcaggttctgtctcctttcttttttttttttttttcttttctttttttggtttctctgtgtagttttggagcctatcctggcactagctctgaagagcaggctggcctcgaactcagagatcagccagcctctgctgggattaaaggtatgcaccaccaacgccccaggttttgtctcaaacaaacaaaaataacatcaggaggtggtgagagggctcagtggtcacaggcattctctgcaaagcctgactctgagttggatcctttagagctacatggttgagggaagagaactgactcctggaaggtgtcctctggtccccacacatagctatacacagcatgtgcattcacacacactaaataatgctatttttaaaaaaattaaaaacaacaacagtttgggttgtgaaaactagaactagataataggtaagaatcaagtatcatgtaaatttgctttcaactcatcccaaaatttgttttatatttcagtttttttccttcctagcttgactgtggagtcttgtccggaagcaaatagttcctttgcagatcccacatgtggacaccggacagtaggtcctcaaatgctccttattaggttggttcaataatatcaattgtttgttactaggcagtgatgttgtacatctggaggagatctcttgagcccataatcaggttattaggaataaatactctaaggctaaaaatgtagcttagtgataagagtgcttgcctggtgtgctgagaccctcggttccatctccacaaccccatattccattacaaaataccttttcaccgtccctagcattaagaaacaaaacaacaaagaagtttttctttcttctgagatcctgcccggagaggcatttaaaactggccagggccaaaaaaaaaaaaaaaaaaagaaaaaaaagaaaagaaaacaggctagggccggcatggtggcgcacgcctttaatcccagcacgcaggaggcagaggcagggcggatctctgtgagtttgaggtcagcctggtctacctagtgagtttcagggcacccagggctaaagagactgtctcaaaaacaaaacagccacacaatcagaaccacagcaaaacgcagttatgatccttggaactgtaggaatgacaagcatttaaataataggacgagccatttttgagaagctctgatttcacaagtgtcagggatgggctctgggcgagtaagattgctaatgctggcctctaaatgagaccacgtggagttgattagattcttttcatgttcctcgtgctctatcaaataactgtacccaaatacacacacacacacacacacacacaatgcgcgcacacacaaaatccttttttagcttaagaagcccagaatcagaagtaaagctaactgtgggacttaagtattattctgaacggaactcccagggcgtgaagcgcgcttcaggcttccagagaagcagctggcgctggatggaatgaaccaagaggccagcacaggggcagatccgtcgagctctcggccaccgagctgagcccttaggttctggggctgggaagggtccctaggattgtgcacctctcccgcgggggacaagcaggggatggcggggctgacgtcgggaggtggcctccacgggaagggacacccggatctcgacacagccttggcagtggagtcaggaagggtaggacagattctggacgccctcttggccagtcctcaccgccccacccccgatggagccgagagtaattcatacaaaaggagggatcgccttcgcccctgggaatcccagggaccgtcgctaaattctggccggcctcccagcccggaaccgctgtgcccgcccagcgcggcgggaggagcctgcgcctagggcggatcgcgggtcggcgggagagcacaagcccacagtccccggcggtgggggaggggcgcgctgagcgggggcccgggagccagcgcggggcaaactgggaaagtggtgtcgtgtgctggctccgccctcttcccgagggtgggggagaacggtataaaagtgcggtagtcgcgttggacgttctttttcgcaacgggtttgccgtcagaacgcaggtgagtggcgggtgtggcctccgcgggcccgggctccctcctttgagcggggtcgggccgccgtgcgggtgtcgtcggccgggcttctctgcgagcgttcccgccctggatggcgggctgtgcgggagggcgagggggggaggcctggcggcggccccggagcctcgcctcgtgtcgggcgtgaggcctagcgtggcttccgccccgccgcgtgccaccgcggccgcgctttgctgtctgcccggctgccctcgattgcctgcccgcggcccgggccaacaaagggagggcgtggagctggctggtagggagccccgtagtccgcatgtcgggcagggagagcggcagcagtcgggggggggaccgggcccgcccgtcccgcagcacatgtccgacgccgcctggacgggtagcggcctgtgtcctgataaggcggccgggcggtgggttttagatgccgggttcaggtggccccgggtcccggcccggtctggccagtaccccgtagtggcttagctccgaggagggcgagcccgcccgcccggcaccagttgcgtgcgcggaaagatggccgctcccgggccctgtagcaaggagctcaaaatggaggacgcggcagcccggcggagcggggcgggtgagtcacccacacaaaggaagagggccttgcccctcgccggccgctgcttcctgtgaccccgtggtgtaccggccgcacttcagtcaccccgggcgctctttcggagcaccgctggcctccgctgggggaggggatctgtctaatggcgttggagtttgctcacatttggtgggtggagactgtagccaggccagcctggccatggaagtaattcttggaatttgcccattttgagtttggagcgaagctgattgacaaagctgcttagccgttcaaaggtattcttcgaactttttttttaaggtgttgtgaaaaccaagctt

SEQ ID NO: 3 - DNA, 4183 bp, Cricetulus griseus
ccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactaccttcatctgggtgagttcagttgatttgtcttgagcctttggggtttacacaagtaaatgacatcatacagttagtgtattgttagtgaatattaatatatgaggcaggctttgctctagcaattttagaactagttttcaggaaagggttcatcttgtgcattggatgtttgattctatcacttagagtttaaactgaaagtgctcaagaggttttatttaggctgggatataaataagcctttctgtagcttgtaatggtatcaggaatttaaaaggccatctggggcacaaagattaagcagaaaaggtagaaggtgagattgggggactttgagtacttcacacactttaatgtgtgagtgctttagtgcatatagtacaactgccagataagggcatccacatctgattgtttggaaggcaccttgtggtttctgggaattcagaattgggagaaaaatgctcccaaccgctgaagcccttggtaatctgcagggtgtttatttagcaggagataaggacaaaaagttatagtgtggagttggttgagttggtagatgtcattacaacaggtggtcttaaattgggttaggagtcactttgaaatacctgggccataagcaaagtggcattttcacctttcaggagaaactggtacacttatccattctatagtgcatgcttgttcaattgggctgatgactaaaccggtgactaaaggtttgtcagtataaatggatgggttgtaggcagacggtgaggaattactatacctgcaaggagtcattgcctgatctgcctggaaaggggcaggattgagtctcagaacgtgtacaccataggatatggaaaaatttgtcacgcctagcattcaacttagtggtgtagcgccacctactggcactttaaaagcttagcatagaggagcatgtgtgttaggagctcggatgggatccagggcctcaaggtttgcatgtaaataaaagccctttaccaaattaactacataccagcatacatcagtcctttagtgttgaaaaacagaagggaaagctaatatatatagtgcttgctttatttaagtctagctgattacgtgtttggttgccagtgtgactagtctggagttgaatttgtcctcagacacgtaaaatggaatttgggattcacaacactctagtatgagggacctaatggcctgtaccaggcacaaacgtgtctataaactacacaaaacgaaggaatttacaggaattaggaaggtattcttaacattaaaacattatgggcattttaaaaaaagctttgacaggatttctttgtcatggctgtcctggagctagttgtgtagaccaggctgggctgaaatcttgtctgcctgcctggcttggacacttttttattatgtatacaacattctgcttccatgtatatctgcacattagaagacggcaccagatctcctaatggatggttgtgagccaccatgtggttgctgggaattgaactcaggacctctggaagagcagtgctcttaacctctgagccatctccagccccagcttgggcacatttttaatggctgggaaatcaaaccccctaggccttctgtcagtaatgaagggcttttggctaccgagagtaggatttaaggttattcggagctgcaggtctgcctcagtgcaggtttgggagtccagcatcttagaaaatgcagtgaagccaagctgagctatattttgtttaaaaaaaaaataagtgggtaaagtgctgctgagcctgatgaccaagctgggacacaagtagaagaacataggccaatgctctatattaaaagcatgggtcatttttaatgctcttgagaaggctatgcctacactactctcagccaccgcagcgtgtttaaattaaactagtttggaaattttctttgggggtaagctatttaacctagtgccttggcaggtatactactgaactctcctcctcattcctttttgttttttaagaatttcagtcaggctcaggcagcccttaaacttgtgattaagcctgagaacagttacgattatgagcctattagtataccgatcaatatgtgaatttttttgggatgggggtcaggcctccctgcctcccaaatactgggactaaaggctgcaccaccacaacctggctcttgaaatacttttctacattttttggggggcatgggtgggagagcagggtttctctgtattagccctggctctcctggaactctgtagaccaggctattcttgagctcagattagcctgtctctgcctcctaaattctgggattaaaggtgtgtgctactgctgcctggctacaaagacatttttttttttcttaaatttaaaaacaaaagtggttcttttagaagggtggttggtgttggcacatactccaagcactcaggttttgagtttgtcccaggaatgaagactgcattactgccgcccctccctggtaagggctacacagagaaatcctatttggagcctatcctggtaactcgctctgtagaccaggctggcctcgaactcaagagaaccacctgcctctgaatgctggtattaagggcaggcaccaccaacacccagcctaaaaaatgtcttttttttaaagatttttttttttttttttacagaataaacattctgtttacaatattctgcttctatgtatatctgcacactagaagagggcacccgatctcataatggatggttgtgagccaccaagtggttgctgggaattgaactcagaacctctggaagagcagtcagtgctcttaacctctgagccatctctccagcccctaaaaatggctcttgagatagggtctcaagtagtttgagactgagttggctatataaacaaggctggcacatagcaccatgtacagctgggtttagtttacatggggtgtttttgtctctggaggcaggaggatcatttgagcatagggagttaatagtgaggtcatgttttatctactcttctgaattgagaactaagctgatgcaaagcaagtttgactgaagaagtccagtttatgagaacaagggtggaaactaatgtgtcaaagatggccttgcatgtgttttagatgatgacccagtcacttgggaattactggatgtgtaagacctatatcttgacaggagtgaacagtgtcttataggtcctatatgaaagaaatgagacatacccattttgtttcccctaagaattcacttttcctaacctggttcatgctatttaggttattttacttgcaaatcctaggtgctcccttacccagtattgcttatgtggcaccaaagtcactcactcccatgatttgcaagtctctgggaacttccatgacaacctagaatagcaactcaaatacattttctcagtaccaattttgaagaaaaaatattttgcaaaatagctgtatggatgggtactaaatagtgaggttatctccagaaggcctatgaagaattaaggttgagttcagttgagttcagcagcaagtttaaggttcatccatttttgtacagtgttttcctattacggtaagtgttttgcctgcaggaatatctgtaccacatgcttgcctggtacctatatcggccagaagagggctttggatcctctggacttgaattacagatgggtattagccaccatttaggtgctgggaattgaaaccaagtcctctggaagaacagcaagtgatcgagtcgac

SEQ ID NO: 4 - DNA, 56 bp, Artificial
  synthetic nucleotide
aagcttcaagttatgctctagaatccggtacctcgagaaaatgcatggcagtcgac

SEQ ID NO: 5 - DNA, 5823 bp, Artificial
  synthetic nucleotide
aagcttgccgccaccatgaaagtgttgagtctgttgtacctgttgacagccattcctggtatcctgtctgacgtacagcttcagcagtcaggacctggcctggtgaaaccttctcagtctctgtccctcacctgcactgtcactggctactcaatcaccagtgattctgcctggaactggatccggcagtttccaggaaacagactggagtggatgggctacataagctacagtggtagcactagctacaacccatctctcaaaagtcgaatctctatcactcgagacacatccaagaaccagttcttcctgcagttgaactctgtgactactgaggacacagccacatattactgtgtaagagggctccggtttgcttactggggccaagggactctggtcactgtgagctcagctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgagggaggcagagcgggtcccactgtccccacactggcccaggctgtgcaggtgtgcctgggccacctagggtggggctcagccaggggctgccctcggcagggtgggggatttgccagcgtgtccctccctccagcagcagctgccctgggctgggccacgggaagccctaggagcccctggggacagacacacagcccctgcctctgtaggagactgtcctgtcctgtgagcgccctgtcctccgaccccccatgcccactcgggggcatgcctagtccatgtgcgtagggacaggccctccctcacccatctacccccacggcactaacccctgtctgccctgcccagcctcgcacccgcatggggacacaaccgactccggggacatgcactctcgggccctgtggagggactggtgcagatgcccacacacacactcagcccagacccgttcaacaaaccccgcactgaggttggccggccacacggccaccacacacacacgtgcacgcctcacacacggagcctcacccgggcgaaccgcacagcacccagaccagagcaaggtcctcgcacacgtgaacactcctcggacacaggcccccacgagccccacgcggcacctcaaggcccacgagccgctcggcagcttctccacatgctgacctgctcagacaaacccagccctcctctcacaaggtgcccctgcagccgccacacacacacaggggaacacacgccacgtcgcgtccctggcactggcccacgtcccaatacagcccttccctgcagctggggtcacatgaggggtgggtttcaccatcctcctgccctctggggctcagggagggacacgggagacggggagtgggtcctgctgagggccaggtcgctatctagggccgggtgtctggctgagccccagggccaaagctggtgcccagggtggacagcttccgggagctgacctcaggacattgttggcccatcccggccgggccctacatcctgggccccgccacagagggaatcacccccagaggcccaagcccagggggacacagcactgaccacccccttcctgtccagagctgcaactggaggagagctgtgcggaggcgcaggacggggagctggacgggctgtggacgaccatcaccatcttcatcacactcttcctgctaagcgtgtgctacagtgccaccgtcaccttcttcaaggtcggccgcacgttgtccccagctgtccttgacattgtcccccatgctgtcacacactgtccctgacactgtcccccactctctccacaaactgtccctgacactgtcccccatgctgtacacacctgtccaacagtgtcccccaggctgtctccacatgtccgacactgtcccccatgctatcccccatcctgacactgtcccccacactgtccccacctgtccttgatgctgtgccccacggtctccccacctctccctgattctgtcccccacgctgtacctgtccccaatgctctcccccagcctgtccccaacactgtccgccatgctgtcccacctttcccccacactgtcccccagtctccgcctgtccctgacgctgtcccacatgcggtcccccattcctgaaactgtcccccatgctgtccccaccttccccaacactgtaccccacagtccccaccttccccaacactgtccccacctgtccctgacactcccccatgctgtccccacctgtcccccactcaccccccacactgtccctacctgtctctgacactgtcccccatgctgtccgcacctgtccccgtcccccacgctgtccctacctgtccctgacactgtcccccatgctgtcccccactttccccagctttccctgtcccccacgctgtccctacctgtccctgacactgacccccatgctgtccgcacctgtcccccactctccccacctctccctgtcacccacgctgtccctacctgtccctgacactgtcccccatgctgtccccacctgtcccccaatctccccacctctccctgtcccccacgctgtccccacctgtccctgacgttgtcttctgtgttatccacatgctgtcactaccatggctctgctctccatgtccaggcctgggggcaggcagtgtagagccccggtacctgggtggcctgaaagacagatgggccttgggagcagggctgtggcctgggtggcctgaggggtgggtggggctcgggggcagggctgtggcctcgctcacccctgtgctgtgccttgcctacaggtgaagtggatcttctcctcagtggtggacctgaagcagaccatcgtccccgactacaggaacatgataaggcagggggcctagggccaccctctgcggggtgtccagggccgcccagaccccacacacgagccgtgggccatgctcagccaccacccaggccacacctgccccctgacctcaccgccctcaaccccatggctctctggcttcgcagtcgccctctgagccctgaaacgccccccttccagaccctgtgcatagcaggtctaccccagacctccgctgcttggtgcatgcagggcgctgggggccaggtgtcccctcagcaggacgtccctgccctctggaccaccaggtgctcacacaaaaggaggtaaccggcatcccaggcccccactcaggcaggacctcgccctggagccaactccgtccacgccagcctcctgaacacaggcatggtttccagatggtgagtgggagcatcagtcgccaaggtagggaagccacagcaccatcaggccctgttggggaggcttccgagagctgcgaaggctcactcagacggccttcctcccagcccgcagccagacagcctccattccgggcactcccgtgaactcctgacatgaggaatgaggttgttctgatttcaagcaaagaacgctgctctctggctcctgagaacagtcccggtgccagcaccaccccttggctgtctgcccacactgctggattctcgggtggaactcgacccgcagggacagccagccccagagtccgcactggggagagaaggggccaggcccaggacactgccacctcccacccactccagtccaccgagatcactcagagaagagcctgggccatgtggccgctgcaggagccccacagtgcaagggtgaggatagcccaaggaagggctgggcatctgcccagacaggcctcccacagaaggctggtgaccaggtcccaggcgggcaagactcagccttggtggggcctgaggacagaggaggcccaggagcatcggggagagaggtggagggacaccgggagagccaggagcgtggacacagccagaactcatcacagaggctggcgtccagtcccgggtcacgtgcagcaggaacaagcagccactctgggggcaccaggtggagaggcaagacgacaaagagggtgcccgtgttcttgcgaaagcggggctgctggccacgagtgctggacagaggcccccacgctctgctgcccccatcacgccgttccgtgactgtcacgcagaatccacagacaggaagggaggctcgagcgggactgcggccagcgcctgcctcggccgtcagggaggactcctgggctcactcgaaggaggtgtcaccatttcagctttggcttttcttcttcttttaaattttctaaagctcattaattgtctttgatgtttcttttgtgatgacaataaaatatcctttttaagtcttgtacttcgtgatgggagccgccttcctgtgtccacgcgcctcctgcccccggtgggaagcacggtcaggaggaggctggtccagctgcacctcgggggctccctgcactcgccccccgcctcctgcagccacacgcattgcccgagcggccctccctggcccctgtcgctacatggacccctggggcttctcctcttttctacatggatgcagtttctcctcctgctgggcacggtgctgcctgccctggtcactctgcgggggacagggcctccagggaaagctgggtcgaggctgggagctggctcaggctggccaggcagagccacagggagggccttccagaaccaaccatggtgtgaagcgagaggtgggtgtcagatctgtgtgcgtcagctcaggaccacagcggggcagctcccacggcagacatggatccactagttctaga

SEQ ID NO: 6 - DNA, 1380 bp, Mus musculus
  CDS (1)..(1380)
atgaaagtgttgagtctgttgtacctgttgacagccattcctggtatcctgtctgacgtacagcttcagcagtcaggacctggcctggtgaaaccttctcagtctctgtccctcacctgcactgtcactggctactcaatcaccagtgattctgcctggaactggatccggcagtttccaggaaacagactggagtggatgggctacataagctacagtggtagcactagctacaacccatctctcaaaagtcgaatctctatcactcgagacacatccaagaaccagttcttcctgcagttgaactctgtgactactgaggacacagccacatattactgtgtaagagggctccggtttgcttactggggccaagggactctggtcactgtgagctcagctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 7 - protein, 460 aa, Mus musculus
MKVLSLLYLLTAIPGILSDVQLQQSGPGLVKPSQSLSLTCTVTGYSITSDSAWNWIRQFPGNRLEWMGYISYSGSTSYNPSLKSRISITRDTSKNQFFLQLNSVTTEDTATYYCVRGLRFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - DNA, 1054 bp, Artificial
  synthetic nucleotide
aagcttaccgccacatggagacagacacaatcctgctatgggtgctgctgctctgggttccaggctccactggtgacattgtacttacacagtctccagcttctttggctgtgtctctagggcagagggccaccatatcctgcagagccagtgaaagtgttgatagttatgtcaatagttttctccactggtaccagcagaaaccaggacagccacccaaactcctcatctatcgtgcatccaacctacaatctgggatccctgccaggttcagtggcagtgggtctaggacagacttcaccctcaccattaatcctgtggaggctgatgatgttgcaacctattactgtcagcaaagtaatgaggatccgtacacgttcggaggggggaccaagctggaaataaaacggactgttgcggcgccgtctgtcttcatcttcccgccatctgatgagcagttgaaatctggaactgcctctgttgtgtgcctgctgaataacttctatcccagagaggccaaagtgcagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcgcataaccggatccactagttctagag

SEQ ID NO: 9 - DNA, 714 bp, mus musculus
  CDS (1)..(714)
atggagacagacacaatcctgctatgggtgctgctgctctgggttccaggctccactggtgacattgtacttacacagtctccagcttctttggctgtgtctctagggcagagggccaccatatcctgcagagccagtgaaagtgttgatagttatgtcaatagttttctccactggtaccagcagaaaccaggacagccacccaaactcctcatctatcgtgcatccaacctacaatctgggatccctgccaggttcagtggcagtgggtctaggacagacttcaccctcaccattaatcctgtggaggctgatgatgttgcaacctattactgtcagcaaagtaatgaggatccgtacacgttcggaggggggaccaagctggaaataaaacggactgttgcggcgccgtctgtcttcatcttcccgccatctgatgagcagttgaaatctggaactgcctctgttgtgtgcctgctgaataacttctatcccagagaggccaaagtgcagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 10 - protein, 238 aa, mus musculus
METDTILLWVLLLWVPGSTGDIVLTQSPASLAVSLGQRATISCRASESVDSYVNSFLHWYQQKPGQPPKLLIYRASNLQSGIPARFSGSGSRTDFTLTINPVEADDVATYYCQQSNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC